SEQUENCE LISTING (10 sequences)

SEQ ID NO: 1 - protein, 8 aa, HomoSapiens
  MOD_RES (1)..(1): biotinylated at N-terminus
RPPQTLSR

SEQ ID NO: 2 - protein, 11 aa, HomoSapiens
  MOD_RES (1)..(1): biotinylated at N-terminus
NLSPDGQYVPR

SEQ ID NO: 3 - protein, 15 aa, HomoSapiens
  MOD_RES (1)..(1): biotinylated at N-terminus
SANAEDAQEFSDVER

SEQ ID NO: 4 - protein, 11 aa, HomoSapiens
  MOD_RES (1)..(1): biotinylated at N-terminus
NFHQYSVEGGK

SEQ ID NO: 5 - protein, 6 aa, HomoSapiens
  MOD_RES (1)..(1): biotinylated at N-terminus
LERPVR

SEQ ID NO: 6 - protein, 15 aa, HomoSapiens
  MOD_RES (1)..(1): biotinylated at N-terminus
VFAQNEEIQEMAQNK

SEQ ID NO: 7 - protein, 10 aa, HomoSapiens
  MOD_RES (1)..(1): biotinylated at N-terminus
DLPLLIENMK

SEQ ID NO: 8 - protein, 12 aa, HomoSapiens
  MOD_RES (1)..(1): biotinylated at N-terminus
ETYGEMADCCAK

SEQ ID NO: 9 - protein, 13 aa, HomoSapiens
  MOD_RES (1)..(1): biotinylated at N-terminus
FIMLNLMHETTDK

SEQ ID NO: 10 - protein, 20 aa, HomoSapiens
  MOD_RES (1)..(1): biotinylated at N-terminus
DLVTQQLPHLMPSNCGLEEK

(19) United States
(12) Patent Application Publication
(10) Pub. No.: US 2004/0253636 A1
(43) Pub. Date: Dec. 16, 2004

(54) Method of protein analysis

(76) Inventors: Mikhail Soloviev, Berks (GB); Jonathan Alexander Terrett, Berks (GB)

Correspondence Address: KLAUBER & JACKSON, 411 HACKENSACK AVENUE, HACKENSACK, NJ, 07601

(21) Appl. No.: 10/490,911

(22) PCT Filed: Sep. 27, 2002
(86) PCT No.: PCT/GB02/04364

Related U.S. Application Data
(60) Provisional application No. 60/326,177, filed on Sep. 27, 2001.

(30) Foreign Application Priority Data
Sep. 27, 2001 (GB) 0123295.8

(51) Int. Cl. G01N 33/53

(52) U.S. Cl. 435/7.1

(57) ABSTRACT

The invention provides a method for the analysis of proteins, in particular complex mixtures of proteins such as those in a biological samples, comprising: a) treating the protein mixture to produce a mixture of peptides; b) contacting the mixture of peptides with at least one amino acid filtering agent that binds to the side chain of an amino acid; c) depleting the micture of those peptides that bind to the filtering agent; d) identifying one or more peptides remaining in the depleted mixture. The method facilitates analysis by decreasing the complexity of a mixture prior to the application of an analytical technique such as mass spectrometry.

This invention relates to methods for compositional analysis of a sample e.g. a biological sample, especially suitable for use in proteomics. In particular, the invention permits a reduction in complexity of a sample, e.g. a biological sample comprising a complex protein mixture, prior to analysis.

Characterization of the complement of expressed proteins from a single genome is a central focus of the evolving field of proteomics. Since one genome produces many proteomes (hundreds in multi-cellular organisms) and the number of expressed genes in a cell is minimally 10,000, the characterization of thousands of proteins to evaluate proteomes can only effectively be accomplished using a high-throughput, automated process.

Generally, proteomics is based on two-dimensional (2D) gel electrophoresis. This technique resolves complex protein mixtures first by isoelectric focusing, using carrier ampholytes and/or immobilised pH gradients, followed by separation according to size using polyacrylamide gel electrophoresis under denaturing conditions. Separated proteins can be identified by their unique position on the 2D gels and quantified using gel imaging systems.

Protein identification can be confirmed using mass spectrometry techniques. The 2D gel-separated proteins are excised and digested (typically with trypsin). The resulting peptides are typically identified using matrix-assisted laser desorption ionization-time of flight (MALDI/TOF) mass spectrometry techniques followed by database mass matching. Further confirmation can be obtained using tandem mass spectrometry (MS/MS) techniques with collision-induced dissociation (CID) to fragment the peptide enabling an amino acid sequence to be generated.

2D gel based proteomics has been applied for proteome-wide expression profiling, as described in U.S. Pat. No. 6,064,754 and U.S. Pat. No. 6,278,794. Pre-fractionation of complex protein mixtures prior to 2D gel separation improves the technique and allows for lower abundant proteins to be separated and identified. More recently one or both dimensions of electrophoretic separation have been substituted with chromatography (Davies et al., Biotechniques 1999, 6:1258-61; Senior, Mol. Med. Today 1999, 5:326-327; Gygi et al, Nature Biotechnology 1999, 17:994-999; Wall et al, Anal. Chem. 2000, 72:1099-111), providing an alternative approach, using the same basic principle of protein separation in more than one dimension followed by protein identification using mass spectrometry.

These techniques are now widely available, but have limitations. Protein staining of gels is biased towards highly abundant proteins. Moreover these techniques are limited by gel/column capacity which can result in missing the less abundant proteins. Additionally, in the above techniques for the separation of complex protein mixtures (with the exception of isoelectrofocusing and chromatofocusing), the purity of final preparations is inversely proportional to the quantity of the materials obtained. This means that larger amounts of highly complex protein mixtures and more purification steps (or separation dimensions) are required in order to yield enough material of sufficient purity for subsequent mass spectrometry (or other) applications.

Recently, another technology has been applied to proteomics research. This technology employs arrays of affinity ligands (antibodies or other agents) immobilised on a variety of solid supports (Soloviev, Drug Discov Today 2001, 6(15):775-777; Arenkov et al, Anal. Biochem. 2000, 278(2):123-31;Vasiliskov et al, Biotechniques 1999, 27(3):592-4, 596-8; Zlatanova et al., Methods Mol. Biol. 2001, 170:17-38; Zhu et al, Nat Genet 2000, 26(3):283-9; Haab et al, Genome Biol. 2001, 2(2):RESEARCH0004; MacBeath and Schreiber, Science 2000, 289:1760-1763; Huang et al, Anal Biochem. 2001, 294(1):55-62). Using arrayed affinity ligands avoids the need for protein separation, as all of the spotted reagents are spatially separated and their positions known. The use of fluorescently labeled protein mixtures further simplifies protein detection and quantitation. Further increases in protein array sensitivity and signal-to-noise ratio have been reported using time resolved fluorescence (Luo and Diamandis, Luminescence 2000, 15(6):409-13) and planar waveguides as protein immobilisation substrates (Weinberger et al, Pharmacogenomics 2000, 1(4):395-416; Pawlak et al., Faraday Discuss. 1998, 111:273-88). However, unlike DNA chips, protein chip based proteomics faces significant difficulties due to the much more heterogeneous character of proteins compared to nucleic acids. A whole cell protein repertoire is extremely complex. Different proteins require different solubilization and separation techniques. Current state of the art in the protein biochemistry has not yielded universal solubilization and affinity assay conditions applicable to all cellular proteins, e.g. small and large, hydrophobic and hydrophilic, soluble and membrane associated, basic and acidic proteins. This significantly limits the applicability of affinity-based chips to small subsets of cellular proteins having very similar physical characteristics.

The recent development of chip-based “peptidomics” provides one significantly better approach to solving this problem (see WO 02/25287). Peptidomics microarrays reduce the complexity of protein binding assays by providing a uniform, standardized binding system based on interactions of capture agents with peptides. Peptides bind to capture agents (or binding partners) with relatively uniform kinetics and affinity (with some variation due to amino acid sequence), whether those binding partners are other peptides, antibodies, receptors, proteins, or even nucleic acids. In this respect, peptidomics microarrays can have binding features more like those associated with nucleic acid hybridization arrays, and thus provide robust, standardized systems for detecting and, optionally, quantifying the total amount of a particular protein present. Peptidomics arrays detect peptides derived from cellular proteins, thus avoiding binding complexities of proteins.

A major drawback of any of the chip-based techniques, however, is the availability and cost of specific capture agents. Unlike nucleic acids, which are both information carriers and perfect affinity ligands, every protein requires the production of its own unique affinity reagent (e.g. an antibody) the development of which, unlike the synthesis of an oligonucleotide or purification of a PCR product, requires significant amounts of time and resources.

Other approaches to protein analysis that permit a reduction in sample complexity and which are not biased towards the abundance of a protein are the isotope-coded affinity tag (ICAT) strategy (Gygi et al, Nature Biotechnology 1999, 17:994-999; WO 00/11208) and a solid phase isotope tagging method which is comparatively simpler, more efficient and more sensitive than the former approach (Zhou, H. et al., 2002 Nature Biotech. 19:512-515). In these techniques the peptide sequence is generated by selecting ions of a particular mass-charge ratio using the MS/MS mode; the sequence is then database searched to reveal the identity of the parent protein. These methods automatically preclude obtaining any information related to peptides that do not contain cysteine residues. This can be an important issue when information about for e.g. post-translational modifications (PTMs) is required. Another option is to differentially label undigested sample using phosphoprotein isotope-coded affinity tag reagents (PhIAT) that combine stable isotope and biotin labelling to enrich and quantitatively measure differences in the O-phosphorylation state of proteins (Goshe, M. et al., 2001, Anal. Chem. 73: 2578-2586). However, these differential labelling methods only permit the enrichment of a selective group of peptides (those containing cysteine residues or phosphorylated residues) leaving the depleted sample remaining highly complex. Any polypeptide lacking these residues will not be detected.

The present invention overcomes the deficiencies of current proteomics techniques, and provides a method that permits qualitative and/or quantitative analysis of peptides and hence proteins in a complex protein mixture; as such it is useful for the proteomic analysis of biological samples. Proteomic analysis using the method of the invention can be used to determine the physiological or biochemical state of a body fluid, a tissue or a cell, where said state includes, but is not limited to, the condition of a cell or tissue after it subjected to a stimulus or is contacted with a molecule, such as a drug, hormone, or other ligand that stimulates or effects cellular activity, after the cell or tissue is partially or completely transformed to become for example, but not limited to, hyperplastic, cancerous, or metastatic, where the cell has entered an apoptotic or other pathway, whether the cell is dysfunctional or diseased, and the type of the cell, i.e. the tissue from which the cell is derived. Proteomics analysis can also be used to determine the protein complement of body fluids or exudates.

Accordingly, the invention provides a method of analysis of a protein mixture, said method comprising:

(a) treating the protein mixture to produce a mixture of peptides;

(b) contacting the mixture of peptides with at least one amino acid filtering agent that binds the side-chain of an amino acid;

(c) depleting the mixture of those peptides that bind to the filtering agent; and

(d) identifying one or more peptides remaining in the depleted mixture.

Generating peptide fragments from proteins ordinarily creates a mixture of tremendous complexity because each protein in a sample yields multiple peptide fragments. The method of the invention permits specific depletion of the peptide mixture i.e. removal of peptide fragments containing specific amino acid residues, thus reducing complexity and facilitating the identification of peptides remaining in the depleted mixture.

Preferably the method results in any unfragmented proteins that remain following step (a) being removed during the filtering and depletion steps (b) and (c).

The method of the invention comprises contacting the mixture of peptides with a reagent that binds the side-chain of an amino acid. Amino acid includes without limitation, the 20 natural amino acids as well as non-natural amino acids known in the art, amino acids comprising PTMs and chemically modified amino acids. A side-chain of an amino acid includes the side-chains of the 20 naturally occurring amino acids as well as modified and non-natural amino acids and amino acids with post-translational modifications (PTMs) or chemically modified residues. In the context of the invention, an amino acid filtering agent includes any compound that is capable of interacting, e.g. binding to a peptide, by recognizing at least one amino acid side chain of the peptide. An amino acid filtering agent may bind to any part of an amino acid side-chain. The interaction is preferably as specific as possible, i.e. without substantial cross-reaction with other amino acid side-chains usually present in a peptide mixture obtained from e.g. a biological sample. Filtering agents which covalently bind to amino acid side-chains are preferred (see Table 1). Alternatively, filtering agents which non-covalently bind may be used. Preferably, the filtering agent is immobilized to a solid support. Various amino acid filter supports can be used, including solid support immobilized chemistries (gels, beads, membranes, etc.), microfluidic devices, such as a multiwell “chip” format for wider scale diagnostics and a LabCD (TECAN, USA) or integrated CD micro laboratory (Amic AB, Sweden) format, and a standard “96 well” (or similar) format for low scale applications. The peptide mixture is preferably contacted with the amino acid filtering agent in solution. The pH of the solution may be adjusted in order to achieve optimal binding of the filtering agent with the selected amino acid side chain.

Any combination of amino acid filtering agents of various specificities or reactivities may be used. Multiple amino acid filters can be contacted sequentially or in parallel with the peptide mixture. Thus in one embodiment, contacting the peptide mixture with an amino acid filter specific for an amino acid is repeated one or more times preferably using a filter specific for an amino acid other than the amino acid targeted in a previous filter step. In another embodiment, a mixture of filtering agents with the same amino acid specificity, or each with different amino acid specificities, or a mixture of reagents with multiple amino acid specificities is contacted with the peptide mixture. Any step can use a single reagent with a single or multiple amino acid specificity. The type and/or number of amino acid filtering agents to use in the method of the invention may be determined with reference to the predicted average size of the peptides in the mixture.

Identification of the peptides remaining in the depleted peptide mixture is preferably performed using mass spectrometry. Peptides remaining in the depleted peptide mixture may be further separated or purified, for example but without limitation, using one or more chromatography steps prior to identification, for example but without limitation, using HPLC. Additionally, peptides captured by the amino acid filter may be released and further separated or purified prior to identification as above.

By selecting one or more amino acid filtering agents, the method of the invention permits a reduction in sample complexity by orders of magnitude. Only a fraction of the original peptides remain in the depleted peptide mixture and are available for analysis. In one embodiment, the amino acid filtering agents or combination of filtering agents are selected such that each protein present in the original protein mixture, e.g. biological sample, is represented in the depleted peptide mixture, such representation being preferably of at least one peptide, more preferably of at least two peptides and most preferably of at least three peptides.

In addition to identifying peptides remaining in the depleted mixture, identification of peptides bound to the amino acid filter may be also performed, e.g. by removing the depleted peptide mixture from the amino acid filtering agent e.g. removing the supernatant. Peptides bound to the amino acid filtering agent are preferably removed from the amino acid filter before identification. Peptides captured by the amino acid filter may be cleaved from the filter enzymatically or chemically.

In a specific embodiment, step (d) of the method of the invention additionally comprises quantifying one or more peptides present in the depleted mixture, preferably using mass spectrometry. Additionally, quantification of one or more peptides which bind to the amino acid filtering agent can also performed, preferably using mass spectrometry.

The present invention is useful for proteomics, pharmacoproteomics, identification of markers of disease, drug target discovery, diagnosis, and in conjunction with therapy. The invention is especially suitable for routine diagnostic applications. Diagnosis includes the measurement or monitoring of protein markers of disease presence, predisposition or progression in an animal and most particularly a human, characterizing, selecting animals or humans for study, including participants in pre-clinical and clinical trials, and identifying those at risk for, or having a particular disorder, or those most likely to respond to a particular therapeutic treatment, or for assessing or monitoring an animal or human response to a particular therapeutic or drug treatment.

The present invention permits the identification and/or quantification of proteins in a biological sample. Any sample that is likely to contain a protein of interest may be analysed. Such biological samples, include body fluid (e.g. blood, serum, plasma, saliva, urine, plural effusions or cerebrospinal fluid), a tissue sample (e.g. a biopsy, blood cells, smears) or homogenates and extracts, including cytoplasm, membranes, and organelles thereof. Cell cultures and culture fluid are also biological samples.

Proteins which may be identified include, without limitation, secreted proteins, integral membrane proteins (including receptors, cell adhesion molecules, and the like), cytoplasmic proteins, proteins from complexes (e.g. ribosomal proteins, polymerase proteins, intracellular signal proteins, etc.), organelle proteins (e.g. mitochondrial proteins, lysosomal proteins, nuclear proteins, endoplasmic reticulum proteins, etc., whether or not membrane associated), and nucleic acid binding proteins (e.g. histones, repressors, transcriptional activators, trans-acting enhancer factors, ribonuclear proteins, etc.). As noted above, an advantage of the invention lies in the detection of peptide fragments of a protein of interest, which reduces or eliminates competitive interactions and anomalous binding resulting from endogenous protein characteristics. Most preferably, the method of the invention permits the identification of a substantial number i.e. most, of the proteins comprising a biological sample.

Samples may be pre-treated to obtain a protein preparation substantially free of unwanted contaminants. Such a treatment may comprise fractionation, differential extraction (membrane and cytosolic fractions); selective depletion (e.g. for removal of albumin, haptoglobin, immunoglobin G); and application to any specific affinity column (e.g. mannose-6-phosphate receptor for lysosomal enzymes; Sleat and Lobel, J Biol Chem 1997, 272:731-8).

Proteins present in a biological sample may be in native form or denatured (Wilkins et al., Biotechnology 1996, 14(1):61-5, e.g. by dissolving in 6M guanidine HCl (or 6-8M urea), 50 mM Tris-HCl (pH8), 2-5 mM DTT (or 2-mercaptoethanol). Proteins present in the sample may also be pre-treated with, e.g. glycosidases to remove glycosylated side-chains, or other means of predictably varying PTMs.

To break disulfide bonds, which link proteins by cysteine residues, and to prevent residues from recombining, a reduction/alkylation step can be performed prior to proteolysis. Dithiothreitol (DTT) may be used for reduction and iodoacetamide may be used for carboxyamidomethylation of cysteine.

The mixture of peptides may be a crude, non-digested mixture of peptides, but is preferably the result of proteolytic digestion of e.g. a biological sample. Reproducible peptide fragments can be generated from biological samples using proteolytic and/or chemical methods or combinations thereof (e.g. Schevchenko et al., Analytical Chemistry 1996, 68:850-858; Houthaeve et al., FEBS Letters, 1995, 376:91-94; Wilkins et al., 1997, Springer ISBN 3-540-62753-7). The sample is thus subjected to conditions that allow enzymatic or chemical cleavage of the individual proteins into peptide mixtures. Preferably, cleavage is a selective enzymatic cleavage, such as but without limitation, using arginine endopeptidase (ArgC), aspartic acid endopeptidase N(AspN), chymotrypsin, glutamic acid endopeptidase C(GluC), lysine endopeptidase C(LysC), trypsin, bromelain, chymotrypsin, ancrod, clostripain, elastase, collagenase, factor Xa, ficin, follipsin, kallikrein, pepsin, thermolysin, thrombin, or V8 endopeptidase. Most preferably, enzymatic cleavage is performed using trypsin. Trypsin digestion is well known in the art. Residual trypsin activity can be inactivated using means known in the art.

Chemical cleavage agents include, but are not limited to, cyanogen bromide, formic acid, HCl, hydroxylamine, N-bromosuccinamide, N-chlorosuccinamide or 2-nitro-5-thiobenzoate.

After digestion of a sample, the peptide mixture generated can optionally be further purified.

Regardless of the type of proteolytic agent used, the optimum digestion time to produce the desired quality of peptide fragments may be determined for example but without limitation, by collecting aliquots every 2 hr and after an overnight digest.

In one embodiment, the biological sample to be quantified can be split into two or more aliquots and each aliquot treated with a different enzyme or chemical agent to produce complementary overlapping target peptide fragments. Each differentially cleaved sample is then subjected to the method of the invention.

Crude peptide mixtures may also be subjected to the analytical methods of the invention in which case the step of proteolysis may be optionally omitted.

Filtering Agents Which Bind Covalently to an Amino Acid

Unmodified peptides as well as proteins generally contain multiple reactive groups. These include seven amino acid specific groups: sulfhydryl groups of cysteines, thioether groups of methionines, imidazolyl groups of histidines, guanidinyl groups of arginines, phenolic groups of tyrosines, indolyl groups of tryptophans and the amino groups of lysines. The method of the invention utilises amino acid side-chain specific chemistries as amino acid filters. In this embodiment a separation of the peptide mixture is thus performed on the basis of the chemical composition of individual peptides rather than on the basis of their sequence or structure.

Examples of the application of amino acid side-chain specific chemistries for binding proteins include the use of acetylimidazole as Tyr-selective reagent (Chun, E, et al., 1963, J. Mol. Biol., 7, 130), mercurial reagents (Bransome, E. and Chargaff, E, 1964, Biochim. Biophys. Acta, 91, 180) or N-ethylmaleimide (Ohno, S, et al., 1964, Chromosoma, 15, 280) as Cys- selective reagents, diketones (Yankeelov J, 1972, Methods Enzymol. 25, 566) and phenylglyoxal (Takahashi K. 1968, J. Biol. Chem. 243:6171-9) as Arg-selective reagents, diethylpyrocarbonate is a selective His-specific compound (Miles E., 1977, Methods Enzymol. 47:431-42). Specific reaction of iodoacetate with methionine was first reported by (Gundlach H., et al., 1959, J. Biol. Chem. 234, 1761) and bromoacetyl compounds for selective immobilisation of Met-containing proteins have been used by The Nest Group, Inc. (Sunnyvale, Calif.) in their commercially available Pi³™-Metionine reagent. Specific chemical binding of tryptophan residues can be achieved using 2-hydroxy-5-nitrobenzyl bromide (Loudon G. and Koshland D. 1970, J. Biol. Chem. 245(9):2247-54).

Table 1 provides a list of preferred chemical reagents for use as amino acid filtering agents and is in no way meant to be limiting.

TABLE 1
Amino acid side-chain specific reagents for use as amino acid filters.

| | Group | | |
|---|---|---|---|
| Reagents | specificity | Crossreactivity | Notes |
| Cys selective reagents | | | |
| α-Haloacetyl compounds | Cys, His, Met, | NH₂— groups (slow | |
| e.g. lodoacetate; α- | Tyr | at low pH) | |
| haloacetamides; | | | |
| bromotrifluoroacetone; N- | | | |
| chloroacetyliodotyramine | | | |
| N-Maleimide derivatives | Cys | NH₂— groups (slow | |
| e.g. N-ethylmaleimide | | at low pH) | |
| (at pH <= 7) | | | |
| Mercurial compounds | Cys | | most specific |
| e.g. p-chloromercuribenzoate | | | |
| (PCMB)/p-hydroxymercuribenzoate | | | |
| (PHMB) in H₂O | | | |
| (optimum at pH 5, competitive | | | |
| displacement possible) | | | |
| Disulphide reagents | Cys | | reversible by β-ME, DTT |
| e.g. 5,5-dithiobis-(2- | | | |
| nitrobenzoic acid) (DTNB); 4,4- | | | |
| dithiodipyridine; methyl-3-nitro- | | | |
| 2-pyridyl disulphide; methyl-2- | | | |
| pyridyl disulphide | | | |
| Tyr selective reagents | | | |
| N-acetylimidazole | Tyr | NH₂— groups (slow) | |
| Diazonium compounds | Tyr, His | NH₂—, Trp, Cys | Optimum at pH 9 |
| | | and Arg—slow | Unstable compounds |
| Arg selective reagents | | | |
| Dicarbonyl compounds | Arg | Lys at pH <= 7 | pH >= 7 |
| e.g. glyoxal; phenylglyoxal; 2,3- | | | |
| butanedione; 1,2- | | | |
| cyclohexanedione | | | |
| His selective reagents | | | |
| p-toluenesulphonylphenyL- | His | | unstable products |
| alaninechloromethyl | | | |
| ketone (TPCK); | | | |
| p-toluenesulphonyllysine- | | | |
| chloromethyl ketone (TLCK); | | | |
| methyl-p-nitrobenzene- | | | cross reactivity is |
| sulphonate | | | limited to Cys |
| Diethylpyrocarbonate | His (at pH4) | NH₂— | reaction reversed |
| | | | at pH >= 7 |
| Met selective reagents | | | |
| α-Haloacetyl compounds | Met at pH3 also | NH₂— groups (slow | |
| | Cys, His, Tyr | at low pH) | |
| Trp selective reagents | | | |
| 2-hydroxy-5-nitrobenzyl | Trp | | |
| bromide (HNBB) | | | |
| p-nitrophenylsulphenyl chloride | Trp, Cys | | |
| Lys selective reagents | | | |
| Sodium nitroprusside | Lys | weak α-amino | |
| | | groups, weak Cys | |
| Glyoxal | | Arg, weak Cys | |

In one embodiment, reagents which bind to carbohydrate moieties present on peptides can be used as amino acid filters, for example using periodate oxidation (see Royer, GP. 1987, Methods Enzymol. 135:141) or by diazonium or phenylisothiocyanate reactions (McBroom, CR. et al., 1972, Methods Enzymol. 28: 212-219).

Filtering Agents Which Bind Non-Covalently to an Amino Acid

Alternatively, or in combination with covalent amino acid filters, complex peptide mixtures may be contacted with agents that recognize and bind in a non-covalent manner with either the amino acid side-chains or with post-translationally or chemically modified amino acids, independently of the sequence or configuration of the peptides. Such agents include but are not limited to, affinity reagents (e.g. antibody, antibody fragments, antibody mimic, CDRs or otherwise derived affinity interactors, including peptides and short nucleic acid fragments) which selectively recognize amino acid side-chains e.g. PTMs; affinity reagents against chemically modified peptides; lectins; ion exchange reagents; hydrophobic and hydrophilic sorbents. In one specific embodiment, depletion of a complex mixture of peptides comprising post-translational modifications such as phosphorylation is performed. Mass spectrometric analysis of phosphopeptides generally requires different conditions to analysis of unphosphorylated peptides. Analysis of both the depleted mixture and phosphorylated peptides which bind to the filtering agent will provide identification of the protein complement of the protein mixture and additional information on individual protein PTMs, respectively. This information may be relevant to e.g. the specific biochemical or physiological state of the cell or tissue sample being analysed.

Specific Antibodies

Affinity reagents, such as antibodies, useful in the context of the present invention, may be generated against single amino acid residues, PTMs or chemical modifications of amino acids. Such antibodies, for example but not limited to, polyclonal or monoclonal antibodies, may be obtained by any standard method known to those skilled in the art.

Polyclonal antibodies that may be used in the methods of the invention are heterogeneous populations of antibody molecules derived from the sera of immunized animals. For example, for the production of polyclonal or monoclonal antibodies, various host animals, including but not limited to rabbits, mice, rats, etc, can be immunized by injection with the native or a synthetic (e.g. recombinant) version of peptides, and the antibodies specific for single amino acids are further selected.

For the preparation of monoclonal antibodies (mAbs), any technique that provides for the production of antibody molecules by continuous cell lines in culture may be used. For example, the hybridoma technique originally developed by Kohler and Milstein (Nature 1975, 256:495-497), as well as the trioma technique, the human B-cell hybridoma technique (Kozbor et al., Immunology Today 1983, 4:72), and the EBV-hybridoma technique to produce human monoclonal antibodies (Cole et al., 1985, in Monoclonal Antibodies and Cancer Therapy, Alan R. Liss, Inc., pp. 77-96).

Also included are antibodies specifically recognizing for example, glutamic acid or phosphotyrosine, phosphoserine, phosphothreonine, phosphohistidine or antibodies recognizing an epitope comprising a specific phosphorylation site or sites. Alternatively, anti-carbohydrate antibodies may be used (Woodward, MP. et al., 1985, J. Immunol. Methods 78:143-153; Galili, U., et al., 1987, Proc. Natl. Acad. Sci. USA 84:1369-1373; Kaladas, PM., et al., 1983, Mol. Immunol. 20:727-735).

Post-Translational Modifications (PTMs)

Amino acid filtering agents may be designed so that the agents recognize and interact with post-translationally or chemically modified residues. Over 250 PTMs that may be utilised in the method of the invention have been described and include: N-formyl-L-methionine; L-selenocysteine; L-cystine; L-erythro-beta-hydroxyasparagine; L-erythro-beta-hydroxyaspartic acid; 5-hydroxy-L-lysine; 3-hydroxy-L-proline; 4-hydroxy-L-proline; 2-pyrrolidone-5-carboxylic acid; L-gamma-carboxyglutamic acid; L-aspartic 4-phosphoric anhydride; S-phospho-L-cysteine; 1′-phospho-L-histidine; 3′-phospho-L-histidine; O-phospho-L-serine; O-phospho-L-threonine; 04′-phospho-L-tyrosine; 2′-[3-carboxamido-3-(trimethylammonio)propyl]-L-histidine; N-acetyl-L-alanine; N-acetyl-L-aspartic acid; N-acetyl-L-cysteine; N-acetyl-L-glutamic acid; N-acetyl-L-glutamine; N-acetylglycine; N-acetyl-L-isoleucine; N2-acetyl-L-lysine; N-acetyl-L-methionine; N-acetyl-L-proline; N-acetyl-L-serine; N-acetyl-L-threonine; N-acetyl-L-tyrosine; N-acetyl-L-valine; N6-acetyl-L-lysine; S-acetyl-L-cysteine; N-formylglycine; D-glucuronyl-N-glycine; N-myristoyl-glycine; N-palmitoyl-L-cysteine; N-methyl-L-alanine; N,N,N-trimethyl-L-alanine; N-methylglycine; N-methyl-L-methionine; N-methyl-L-phenylalanine; N,N-dimethyl-L-proline; omega-N,omega-N′-dimethyl-L-arginine; omega-N,omega-N-dimethyl-L-arginine; omega-N-methyl-L-arginine; N4-methyl-L-asparagine; N5-methyl-L-glutamine; L-glutamic acid 5-methyl ester; 3′-methyl-L-histidine; N6,N6,N6-trimethyl-L-lysine; N6,N6-dimethyl-L-lysine; N6-methyl-L-lysine; N6-palmitoyl-L-lysine; N6-myristoyl-L-lysine; O-palmitoyl-L-threonine; O-palmitoyl-L-serine; L-alanine amide; L-arginine amide; L-asparagine amide; L-aspartic acid 1-amide; L-cysteine amide; L-glutamine amide; L-glutamic acid 1-amide; glycine amide; L-histidine amide; L-isoleucine amide; L-leucine amide; L-lysine amide; L-methionine amide; L-phenylalanine amide; L-proline amide; L-serine amide; L-threonine amide; L-tryptophan amide; L-tyrosine amide; L-valine amide; L-cysteine methyl disulfide; S-farnesyl-L-cysteine; S-12-hydroxyfarnesyl-L-cysteine; S-geranylgeranyl-L-cysteine; L-cysteine methyl ester; S-palmitoyl-L-cysteine; S-diacylglycerol-L-cysteine; S-(L-isoglutamyl)-L-cysteine; 2′-(S-L-cysteinyl)-L-histidine; L-lanthionine; meso-lanthionine; 3-methyl-L-lanthionine; 3′-(S-L-cysteinyl)-L-tyrosine; N6-carboxy-L-lysine; N6-1-carboxyethyl-L-lysine; N6-(4-amino-2-hydroxybutyl)-L-lysine; N6-biotinyl-L-lysine; N6-lipoyl-L-lysine; N6-pyridoxal phosphate-L-lysine; N6-retinal-L-lysine; L-allysine; L-lysinoalanine; N6-(L-isoglutamyl)-L-lysine; N6-glycyl-L-lysine; N-(L-isoaspartyl)-glycine; pyruvic acid; L-3-phenylacetic acid; 2-oxobutanoic acid; N2-succinyl-L-tryptophan; S-phycocyanobilin-L-cysteine; S-phycoerythrobilin-L-cysteine; S-phytochromobilin-L-cysteine; heme-bis-L-cysteine; heme-L-cysteine; tetrakis-L-cysteinyl iron; tetrakis-L-cysteinyl diiron disulfide; tris-L-cysteinyl triiron trisulfide; tris-L-cysteinyl triiron tetrasulfide; tetrakis-L-cysteinyl tetrairon tetrasulfide; L-cysteinyl homocitryl molybdenum-heptairon-nonasulfide; L-cysteinyl molybdopterin; S-(8alpha-FAD)-L-cysteine; 3′-(8alpha-FAD)-L-histidine; 04′-(8alpha-FAD)-L-tyrosine; L-3′,4′-dihydroxyphenylalanine; L-2′,4′,5′-topaquinone; L-tryptophyl quinone; 4′-(L-tryptophan)-L-tryptophyl quinone; O-phosphopantetheine-L-serine; N4-glycosyl-L-asparagine; S-glycosyl-L-cysteine; 05-glycosyl-L-hydroxylysine; O-glycosyl-L-serine; O-glycosyl-L-threonine; 1′-glycosyl-L-tryptophan; 04′-glycosyl-L-tyrosine; N-asparaginyl-glycosylphosphatidylinositolethanolamine; N-aspartyl-glycosylphosphatidylinositolethanolamine; N-cysteinyl-glycosylphosphatidylinositolethanolamine; N-glycyl-glycosylphosphatidylinositolethanolamine; N-seryl-glycosylphosphatidylinositolethanolamine; N-alanyl-glycosylphosphatidylinositolethanolamine; N-seryl-glycosylsphingolipidinositolethanolamine; O-(phosphoribosyl dephospho-coenzyme A)-L-serine; omega-N-(ADP-ribosyl)-L-arginine; S-(ADP-ribosyl)-L-cysteine; L-glutamyl 5-glycerylphosphorylethanolamine; S-sulfo-L-cysteine; 04′-sulfo-L-tyrosine; L-bromohistidine; L-2′-bromophenylalanine; L-3′-bromophenylalanine; L-4′-bromophenylalanine; 3′,3″,5′-triiodo-L-thyronine; L-thyroxine; L-6′-bromotryptophan; dehydroalanine; (Z)-dehydrobutyrine; dehydrotyrosine; L-seryl-5-imidazolinone glycine; L-3-oxoalanine; lactic acid; L-alanyl-5-imidazolinone glycine; L-cysteinyl-5-imidazolinone glycine; D-alanine; D-allo-isoleucine; D-methionine; D-phenylalanine; D-serine; D-asparagine; D-leucine; D-tryptophan; L-isoglutamyl-polyglycine; L-isoglutamyl-polyglutamic acid; 04′-(phospho-5′-adenosine)-L-tyrosine; S-(2-aminovinyl)-D-cysteine; L-cysteine sulfenic acid; S-glycyl-L-cysteine; S-4-hydroxycinnamyl-L-cysteine; chondroitin sulfate D-glucuronyl-D-galactosyl-D-galactosyl-D-xylosyl-L-serine; dermatan 4-sulfate D-glucuronyl-D-galactosyl-D-galactosyl-D-xylosyl-L-serine; heparan sulfate D-glucuronyl-D-galactosyl-D-galactosyl-D-xylosyl-L-serine; N6-formyl-L-lysine; 04-glycosyl-L-hydroxyproline; O-(phospho-5′-RNA)-L-serine; L-citrulline; 4-hydroxy-L-arginine; N-(L-isoaspartyl)-L-cysteine; 2′-alpha-mannosyl-L-tryptophan; N6-mureinyl-L-lysine; 1-chondroitin sulfate-L-aspartic acid ester; S-(6-FMN)-L-cysteine; 1′-(8alpha-FAD)-L-histidine; omega-N-phospho-L-arginine; S-diphytanylglycerol diether-L-cysteine; alpha-1-microglobulin-Ig alpha complex chromophore; bis-L-cysteinyl bis-L-histidino diiron disulfide; hexakis-L-cysteinyl hexairon hexasulfide; N6-(phospho-5′-adenosine)-L-lysine; N6-(phospho-5′-guanosine)-L-lysine; L-cysteine glutathione disulfide; S-nitrosyl-L-cysteine; N4-(ADP-ribosyl)-L-asparagine; L-beta-methylthioaspartic acid; 5′-(N-6-L-lysine)-L-topaquinone; S-methyl-L-cysteine; 4-hydroxy-L-lysine; N4-hydroxymethyl-L-asparagine; O-(ADP-ribosyl)-L-serine; L-cysteine oxazolecarboxylic acid; L-cysteine oxazolinecarboxylic acid; glycine oxazolecarboxylic acid; glycine thiazolecarboxylic acid; L-serine thiazolecarboxylic acid; L-phenyalanine thiazolecarboxylic acid; L-cysteine thiazolecarboxylic acid; L-lysine thiazolecarboxylic acid; O-(phospho-5′-DNA)-L-serine; keratan sulfate D-glucuronyl-D-galactosyl-D-galactosyl-D-xylosyl-L-threonine; L-selenocysteinyl molybdopterin guanine dinucleotide; 04′-(phospho-5′-RNA)-L-tyrosine; 3-(3′-L-histidyl)-L-tyrosine; L-methionine sulfone; dipyrrolylmethanemethyl-L-cysteine; S-(2-aminovinyl)-3-methyl-D-cysteine; 04′-(phospho-5′-DNA)-L-tyrosine; O-(phospho-5′-DNA)-L-threonine; 0-4′-(phospho-5′-uridine)-L-tyrosine; N-(L-glutamyl)-L-tyrosine; S-phycobiliviolin-L-cysteine; phycoerythrobilin-bis-L-cysteine; phycourobilin-bis-L-cysteine; N-L-glutamyl-poly-L-glutamic acid; L-cysteine sulfinic acid; L-3′,4′,5′-trihydroxyphenylalanine; O-(sN-1-glycerophosphoryl)-L-serine; 1-thioglycine; heme P460-bis-L-cysteine-L-tyrosine; O-(phospho-5′-adenosine)-L-threonine; tris-L-cysteinyl-L-cysteine persulfido-bis-L-glutamato-L-histidino tetrairon disulfide trioxide; L-cysteine persulfide; 3′-(1′-L-histidyl)-L-tyrosine; heme P460-bis-L-cysteine-L-lysine; 5-methyl-L-arginine; 2-methyl-L-glutamine; N-pyruvic acid 2-iminyl-L-cysteine; N-pyruvic acid 2-iminyl-L-valine; heme-L-histidine; S-selenyl-L-cysteine; N6-methyl-N-6-poly(N-methyl-propylamine)-L-lysine; hemediol-L-aspartyl ester-L-glutamyl ester; hemediol-L-aspartyl ester-L-glutamyl ester-L-methionine sulfonium; L-cysteinyl molybdopterin guanine dinucleotide; trans-2,3-cis-3,4-dihydroxy-L-proline; pyrroloquinoline quinone; tris-L-cysteinyl-L-N1′-histidino tetrairon tetrasulfide; tris-L-cysteinyl-L-N3′-histidino tetrairon tetrasulfide; tris-L-cysteinyl-L-aspartato tetrairon tetrasulfide; N6-pyruvic acid 2-iminyl-L-lysine; tris-L-cysteinyl-L-serinyl tetrairon tetrasulfide; bis-L-cysteinyl-L-N3′-histidino-L-serinyl tetrairon tetrasulfide; O-octanoyl-L-serine. One of ordinary skill in the art would readily recognize that other PTMs occur and are suitable for binding using the method of the invention.

Examples of alkylation include, but are not limited to, those disclosed in Saragoni et al., 2000, Neurochem. Res. 25:59-70; Fanapour et. al, 1999, WMJ, 98:51-4; Raju et. al, 1997, Exp. Cell Res. 235:145-54; Zhao et al, 2000, Mol. Biol. Cell. 11:721-34; or Seabra, J. 1996, Biol. Chem. 271:14398-404.

Examples of phosphorylation include, but are not limited to, those disclosed in Vanmechelen et. al, 2000, Neurosci. Lett. 285:49-52; Lutz et. al, 1994, Pancreas, 9:418-24; Gitlits et. al., 2000, J. Investig. Med. 48:172-82; or Quin and McGuckin, 2000, Int. J. Cancer, 87:499-506.

An example of sulphation includes, but is not limited to, that disclosed in Manzella et. al, 1995 J. Biol. Chem. 270S:21665-71.

Examples of post-translational modification by oxidation or reduction include, but are not limited to, those disclosed in Magsino et. al, 2000, Metabolism, 49:799-803; or Stief et. al, 2000, Thromb. Res. 97:473-80.

Examples of ADP-ribosylation include, but are not limited to, those disclosed in Galluzzo et. al, 1995, Eur. J. Immunol. 25:2932-9; or Thraves et. al, 1996, Med. 50:961-72.

An example of hydroxylation includes, but is not limited to, that disclosed in Brinckmann et. al, J. Invest. Dermatol. 1999, 113:617-21.

Examples of glycosylation include, but are not limited to, those disclosed in Johnson et. al, Br. J. Cancer 1999, 81:1188-95; Fulop et. al, Biochem. 1996, J. 319:935-40; Dow et. al, Exp. Neurol. 1994, 28:233-8; Kelly et. al, J. Biol. Chem. 1993, 268:10416-24; Goss et. al, Clin. Cancer Res. 1995, 1:935-44; or Sleat et. al, Biochem. J. 1998, 334:547-51.

An example of glucosylphosphatidylinositide addition includes, but is not limited to, that disclosed in Poncet et. al, Acta Neuropathol. 1996, 91:400-8.

An example of ubiquitination includes, but is not limited to, that disclosed in Chu et. al, Mod. Pathol. 2000, 13:420-6.

Examples of methylation include, but are not limited to, those disclosed in Aletta J. et al., 1998, Trends in Biochem. Sci. 23:89-91.

An example of a translocation leading to a disease state includes, but is not limited to, that disclosed in Reddy et. al, Trends Neurosci. 1999, 22:248-55.

“Amino Acid Filter” Formats

Amino acid filters may be used in a variety of formats. Preferred formats include immobilization of amino acid filtering agents on a solid support. Any solid phase support for use in the present invention will be inert to the reaction conditions for binding and is not limited to a specific type of support. Indeed, a large number of supports are available and are known to one of ordinary skill in the art. Solid phase supports include silica gels, resins, derivatized plastic films, glass beads, cotton, plastic beads, alumina gels, magnetic beads, membranes (including but not limited to, nitrocellulose, cellulose, nylon, and glass wool), plastic and glass dishes or wells, etc Polystyrene resin (e.g. PAM-resin, Bachem Inc., PA; Peninsula Laboratories, CA), POLYHIPE™ resin (Aminotech, Canada), polyamide resin (Peninsula Laboratories, CA), polystyrene resin grafted with polyethylene glycol (TentaGel™, Rapp Polymere, Tubingen, Germany) or polydimethylacrylamide resin (obtained from Milligen/Biosearch, CA) are encompassed.

Chemical Cross-Linking Agents

Other examples of reagents suitable for use as amino acid filtering agents include, but are not limited to, homo- or hetero-, bi- or multi-functional reagents. These reagents can be used to recognize and cross-link the recognized peptides facilitating their precipitation or separation by mass or size. According to this embodiment, non-reacted (non-recognized) peptides are separated from the recognized cross-link high molecular weight complexes. Examples of conventional cross-linking agents are carbodiimides, such as 1-cyclohexyl-3-(2-morpholinyl-(4-ethyl) carbodiimide (CMC), 1-ethyl-3-(3-dimethyaminopropyl) carbodiimide (EDC) and 1-ethyl-3-(4-azonia-4,4-dimethylpentyl) carbodiimide.

Examples of other suitable cross-linking agents are cyanogen bromide, glutaraldehyde and succinic anhydride. In general, any of a number of homo-bifunctional agents including a homo-bifunctional aldehyde, a homo-bifunctional epoxide, a homo-bifunctional imidoester, a homo-bifunctional N-hydroxysuccinimide ester, a homobifunctional maleimide, a homo-bifunctional alkyl halide, a homo-bifunctional pyridyl disulfide, a homo-bifunctional aryl halide, a homo-bifunctional hydrazide, a homo-bifunctional diazonium derivative and a homo-bifunctional photoreactive compound may be used. Also included are hetero-bifunctional compounds, for example, compounds having an amine-reactive and a sulfhydryl-reactive group, compounds with an amine-reactive and a photoreactive group and compounds with a carbonyl-reactive and a sulfhydryl-reactive group.

Specific examples of such homo-bifunctional cross-linking agents include the bifunctional N-hydroxysuccinimide esters dithiobis(succinimidylpropionate), disuccinimidyl suberate, and disuccinimidyl tartarate; the bifunctional imidoesters dimethyl adipimidate, dimethyl pimelimidate, and dimethyl suberimidate; the bifunctional sulfhydryl-reactive cross-linkers 1,4-di-[3′-(2′-pyridyldithio) propion-amido]butane, bismaleimidohexane, and bis-N-maleimido-1,8-octane; the bifunctional aryl halides 1,5-difluoro-2,4-dinitrobenzene and 4,4′-difluoro-3,3′-dinitrophenylsulfone; bifunctional photoreactive agents such as bis-[b-(4-azidosalicylamide)ethyl]disulfide; the bifunctional aldehydes formaldehyde, malondialdehyde, succinaldehyde, glutaraldehyde, and adiphaldehyde; a bifunctional epoxide such as 1,4-butanediol diglycidyl ether; the bifunctional hydrazides adipic acid dihydrazide, carbohydrazide, and succinic acid dihydrazide; the bifunctional diazoniums o-tolidine, diazotized and bis-diazotized benzidine; the bifunctional alkylhalides N,N′-ethylene-bis(iodoacetamide), N,N′-hexamethylene-bis(iodoacetamide), N,N′-undecamethylene-bis(iodoacetamide), as well as benzylhalides and halomustards, such as al a′-diiodo-p-xylene sulfonic acid and tri(2-chloroethyl)amine, respectively.

Examples of other common hetero-bifunctional cross-linking agents include, but are not limited to, SMCC [succinimidyl-4-(N-maleimidomethyl)cyclohexane-1-carboxylate)], MBS (m-maleimidobenzoyl-N-hydroxysuccinimide ester), SIAB [N-succinimidyl(4-iodacetyl) aminobenzoate], SMPB [succinimidyl-4-(p-maleimidophenyl)butyrate], GMBS [N-(gamma-maleimidobutyryloxy)succinimide ester], MPHB [4-(4-N-maleimidophenyl) butyric acid hydrazide], M2C2H [4-(N-maleimidomethyl) cyclohexane-1-carboxyl-hydrazide], SMPT [succinimidyloxycarbonyl-alpha-methyl-alpha-(2-pyridyidithio)toluene], and SPDP [N-succinimidyl 3-(2-pyridyldithio) propionate].

Several different amino acid filters may be used in a sequential manner or in parallel, for example by means of a number of interlocked chambers or a combination of amino acid filter-linked beads.

Microfluidic multiwell “chip” formats can also be advantageous for wider scale diagnostics. A LabCD (TECAN, USA) or integrated CD micro laboratory (Amic AB, Sweden) format may be useful as well.

Standard 96-well (or similar) formats are suitable for low scale applications, whereas individual interchangeable amino acid filters could be provided for customized applications.

Depletion Approach

Using the method of the invention, a peptide mixture is depleted in a quantitative and reproducible manner by passing the mixture through an amino acid filter that recognizes a selected amino acid side-chain or chains. In a preferred embodiment, one or more amino acid filters that recognize a selected amino acid side-chain or chains are used, either in combination or consecutively. After separation of the amino acid filter with bound peptides, the depleted peptide mixture contains fewer peptides and as such has been subjected to a reduction in complexity. Preferably, only those peptides that do not contain an amino acid recognized by the amino acid filter or filters used remain in the mixture. These peptides can thus be subjected to MALDI-TOF mass spectrometry or MS/MS analysis for peptide identification. Because the depleted peptide pools will contain peptides of reduced amino acid complexity, this further facilitates the analysis of mass spectra produced by MALDI-TOF mass spectrometry. Preferably, this reduction in the amino acid complexity permits a greater number of peptide peaks to be identified from a mass spectrum. Alternatively, the depleted mixture can be further purified using means known in the art.

For example, but without limitation, the reactive groups present on the side-chains of the seven amino acid specific groups described above allows the use of to use up to seven independent amino acid covalent filters. It is understood that one or more amino acid filters may be used consecutively or in combination and that any one filter may be used more than once. It is also clear that longer peptides on the balance of probabilities can comprise a wider variety of amino acids and conversely shorter peptides can comprise a lesser variety. For example but without limitation, a peptide of twenty amino acids in length could be comprised of one of each of the twenty amino acids. Any one amino acid filter specific for a single side-chain could be expected to deplete a peptide mixture comprising peptides of twenty amino acids substantially, such that more than 80%, and more preferably, 85% or 90% and most preferably 95% of said peptides would be retained by the amino acid filter. In the same way, any one amino acid filter specific for a single side-chain could be expected to deplete a peptide mixture comprising peptides of ten amino acids to a less substantial amount than one comprised of peptides of twenty amino acids in length, said ten amino acid peptides being less probable to comprise an amino acid with a side-chain recognised by an amino acid filter, such that more than 50%, and more preferably, 60%, 70% or 80% and most preferably 90% of said peptides would be retained by the amino acid filter.

Using a combination of all such filters preferably results in a maximum possible depletion i.e. a substantial depletion. In one embodiment, a combination of filters specific for the seven amino acid groups is used to deplete complex peptide mixtures, for example but without limitation, a biological sample comprising a whole cell proteome. The use of individual amino acid filters or subsets of filters is preferred for depleting simpler protein mixtures, which contain fewer individual proteins, for example but without limitation, a biological sample comprising a simple microorganism proteomes, or a biological sample comprising a subfraction resulting from the fractionation of a mammalian whole cell extract. It will be understood by one skilled in the art that the permutations of filters for use can be varied with the sample type selected. Preferably, the permutation of amino acid filters for use is optimized to achieve the desired results for a given sample.

Most preferably, the peptide mixture is been prepared using tryptic digestion. The preparation of a peptide mixture by digestion of a sample comprising proteins with trypsin results in the special case where lysine or arginine are present in every peptide, except the most C-terminal peptide, unless the C-terminal amino acid is lysine or arginine itself. The chances of finding either lysine or arginine in any one tryptic peptide is close to 100%; trypsin does not comprise exoprotease activity thus any protein whose C-terminus is lysine or arginine is an exception. In one embodiment, a sample of interest is digested with trypsin and the resulting peptide mixture treated with amino acid filters recognizing arginine and lysine. The depleted peptide mixture will comprise the C-terminal peptide of any protein which does not comprise a lysine or arginine residue.

Thus, using the method of the invention and a selection of amino acid filters, the complexity of a highly complex sample may be reduced substantially, permitting the identification of a substantial proportion of the proteins present in the original sample.

An advantage of employing amino acid filters based on affinity reagents for the depletion of a peptide mixture instead of with reagents which bind covalently to an amino acid side chain include the use of a larger number of possible filters. In one embodiment, amino acid filters that bind covalently to an amino acid side-chain are used in combination with amino acid filters based on affinity reagents. Unlike amino acid side-chain specific chemistries, which are generally limited to seven amino acids, affinity reagents can be obtained for larger numbers of single amino acids. For example but without limitation, peptide mixtures may also be selectively depleted in peptides containing PTMs by using a filter that recognizes such a modification, e.g. phosphorylation.

In a preferred embodiment, the peptide mixture is passed through the selected amino acid filter which bind peptides containing the recognized amino acid side-chain. Recognized peptides are bound to the amino acid filter via the formation of a bond between the amino acid filter reagent and the amino acid side-chain. The supernatant remaining after removal of the amino acid filter is the depleted peptide mixture and the peptides present in said depleted mixture are identified preferably using mass spectrometry. The amino acid filter is then washed to remove unbound peptides and the peptides released by chemical or enzymatic cleavage in order to free the bound peptides. The protocol can be repeated using one or more amino acid filters. In another embodiment, the method of the invention can additionally comprise selectively enriching for peptides of interest using amino acid filters that bind peptides non-covalently such as filters comprising affinity reagents.

Quantitative Analysis

In addition to the step of depletion using the method of the invention, the peptide mixtures may be subjected to quantitative analysis, preferably using mass spectrometry. This can be using primary mass spectrometry (e.g. MALDI-TOF mass spectrometry) or MS/MS analysis.

In a preferred embodiment, peptides present in a depleted peptide mixture are initially analyzed using MALDI-TOF mass spectrometry with delayed extraction and a reflectron in the time-of-flight chamber. This instrument configuration is used to determine accurately the molecular weights (preferably less than 100 ppm) of modified and unmodified peptides.

The data collected using MALDI-TOF is represented as a list of parent ion masses. Masses due to the presence of the capture agent can be ignored and analysis focused on masses arising from the target peptide fragments. Intensities of each mass (m/z) feature in the mass spectrum are measured by methods known to those skilled in the art e.g. as specified in WO 01/75454.

Where an identification is needed, for example to implement proteomics analysis, further analysis of the sample/matrix spot can be performed using any standard method of MS/MS and in particular using MALDI-TOF/TOF (Applied Biosystems, Framingham, Mass.) or MALDI II Q-TOF (Micromass) or Q-STAR (Sciex) all of which are systems which continue MALDI-TOF with tandem mass spectrometry. This generates a fragmentation spectrum, which can be used to generate sequence information.

Database searching of the primary mass data provided by MALDI-TOF mass spectrometry may be used to identify possible PTMs of peptides. Where there is more than one possible site of a PTM, MS/MS can be used to provide specific information on the site of such PTMs. For example high energy CID provided by MALDI-TOF/TOF mass spectrometry has been shown to unambiguously establish the site of peptide phosphorylation (Analysis of PTMs using a MALDI-TOF/TOF Mass Spectrometer, DeGnore et al. Poster presentation at the 49th ASMS conference on Mass Spectrometry and Allied Topics, Chicago).

In one embodiment, biological samples are labelled with an isotope. In a preferred embodiment, peptides comprise an isotopic label. For example and without limitation, samples e.g. a test and a control sample, can be differentially labelled using stable isotope labelling. In this embodiment, peptides generated by digestion of samples can be differentially labelled, or optionally fractionated prior to or after differential labelling with Do- or D₃-methanol (Goodlett et al., 2001, Rapid Comm. Mass Spectrom. 15:1214-1221). Alternatively, other isotopic labels known in the art can be used. In another embodiment, the mass-coded abundance tagging (MCAT) technique can be used, wherein the ε-amino group of lysine residues of one sample is derivatized with O-methylisourea while the second sample remains underivatized (Cagney and Emili, 2002, Nature Biotech. 20:163-170).

In another preferred embodiment, two or more samples originating from, for example but without limitation, different sets of tissues or cells could be subject to mass spectrometry at the same time. Peptide mixtures are labelled (tagged) with tags of different molecular mass, but with identical or closely matching chemical and physical properties. Most preferably, said tags are present on all peptides in the mixture. This is achieved by utilizing labelling through amino groups, preferably through alpha-amino groups, or alternatively through carboxyl groups, preferably through alpha-carboxyl groups. Examples of amino-group reactive chemistries include but are not limited to, aryl halides, aldehydes, ketones, alpha-haloacetyl, N-maleimide or derivatives of these, as well as acylating reagents. Examples of carboxy-group reactive chemistries include but are not limited to, diazoacetate esters, diazoacetamides and carbodiimides. Peptide mixtures are preferably labelled with tags of different masses either through their amino- or carboxyl-group using tags which comprise side-chain differences. Alternatively, tags which are related and comprise identical side-chains may be used.

Fluorophenyl-isocyanates and fluorophenyl-isothiocyanates are just two of numerous examples of acylating reagents with mass differences introduced through modifying the reagents or their own side chain modifications. The following text indicates examples modifications to acylating reagents and are in no way intended to be limiting. The above acylating reagents can be modified with fluorine, chlorine, bromine or iodine. For example and without limitation, the differential tags for use in differentially labelling two samples could comprise bromine-isothiocyanate vs. iodine-isothiocyanate). Alternatively, but without limitation, these could be mono-, di-, or tri- modifications (e.g. use fluorophenyl-isothiocyanate vs. difluorophenyl-isothiocyanate). It is understood that the amino-reactive chemistry can be modified (e.g. use isocyanate vs. isothiocyanate). Another alternative is to differentially derivatise a tag (e.g. isocyanate vs. phenyl-isocyanates). Preferably small mass differences exist between the differential tags. Alternatively, larger differences can be used.

Quantitative analysis can be accomplished using other techniques as well, which are available by virtue of the reduction in complexity achieved by the invention. In particular, high performance chromatography, capillary electrophoresis, two-dimensional electrophoresis and similar analytical techniques provide for quantitation of individual peptide fragments left after depletion of a preparation. Identification of individual peptides may require other techniques like mass spectrometry (or Edman sequencing), but once the peak is identified, it can be quantitated by measurement of a property such as ultraviolet absorption.

The above techniques can also be used qualitatively as can polyacrylamide gel electrophoresis (PAGE), isoelectric focusing, chromatography (e.g. ion exchange, affinity, immunoaffinity, and sizing column chromatography), centrifugation, differential solubility, immunoprecipitation, or any other standard technique also known for the purification of proteins.

The present invention further contemplates the analysis of a peptide “fingerprint” after depletion, which fingerprint may change as peaks for specific peptides or peptide fragments increase or decease, appear or disappear, depending on the nature of the sample, e.g. the physiological or biochemical state of a cell or organism.

The method of the invention can be customized and various bioinformatics tools can be applied to facilitate throughput. Various types of apparatus, typically microprocessor (i.e. computer) controlled, are available for the quantitation of peptides. In particular, mass spectrometry employs well-known types of apparatus, e.g. as set forth in the references noted above. The invention further specifically contemplates adapting such apparatus for the specific analysis of protein samples according to the invention. In some respects, the robust, standardizable, uniform assays of the present invention permit adaptation of specific features of the apparatus, including but not limited to incubation time, detection parameters, and processing software.

Using all possible combinations of enzymatic/chemical protein digestion methods plus all combinations of the absorption chemistries will permit thousands of individual peptides to be identified, preferably using mass spectrometry. Software packages can be utilised to calculate the best strategy (e.g. the best combination of digestion enzymes and filter combinations) for identification/quantitation of a particular (known) protein in a number of test tissues. Software can also be used to calculate optimal amino acid filter combinations for determining the maximum number of individual peptides after using particular proteolytic digestion techniques.

The present invention greatly facilitates qualitative and quantitative analysis of a complex protein mixture by decreasing the compositional complexity of all peptides derived from the digestion of a biological sample, or selectively and quantitatively enriching certain peptides present in the mixture. The methods of the invention offer good reproducibility, are easy to automate, and can be performed using various customized formats, such as a microfluidic device, or a multi-well format for parallel analysis. Preferably, the method is optimized by using calculated/predicted combinations of digestion/separation for quantitative analysis of known protein(s) by mass spectrometry. As such, the method of the invention is suitable for routine applications.

In a specific embodiment, software specifically evaluates diagnostic supports for the presence and amount of key disease markers. The software processes the detected peptides against a database of known markers for particular cellular conditions, and provides as output, not raw binding intensity data, but a most likely diagnosis. Such an apparatus has clear application in commercial diagnostic laboratories, where the number of samples to be analyzed is large.

The method of the invention has a number of advantages over conventional proteomics such as:

(i) lack of requirement for gels or chromatography;

(ii) more efficient than chromatography—a depleted peptide mixture can be produced in a one step process with little or no dilution; and

(iii) recovery is high and specific.

In one embodiment, the method of the invention can be used as a diagnostic method for a particular protein of interest where, the best strategy is calculated, for example but without limitation, the best combination of digestion enzymes and amino acid filter combinations, for the quantitation of said protein or proteins in a number of test tissues (e.g. a diseased versus a normal sample of tissue, cells, body fluid, etc.).

Preferably, a protein or a peptide that is differently expressed in a disease can be detected in a biological sample and noted as a marker of the disease or change in biochemical status. Examples of such markers include, but are not limited to, Cystatin C for renal dysfunction, (Fliser D. and Ritz E., Am. J. Kidney Dis. 2001, 37(1): 79-83); prostate-specific antigen (PSA) for prostate cancer, (Millenbrand et al., Anticancer Res., 2000, 20(6D): 499-6); Angiotensin II/ACE for heart failure (Kim SD; Biol. Res. Nurs. 2000, 1(3): 210-26).

In another embodiment differential expression can be detected in an experimental sample as compared to a listing or database of previously characterized (either experimentally or theoretically, in silico) samples.

The method of the invention is also useful to quantify multiple proteins whose expression levels best correlate with a physiological or biochemical state, for example, and without limitation, as determined by multivariate analysis of protein expression levels. This physiological or biochemical state may be a response, such as, without limitation, a response to a xenobiotic stress; a hyperplastic, cancerous, or metastatic state; an apoptotic, dysfunctional or diseased state; or a particular phenotype. Central nervous system dysfunctions or diseases, such as depression, schizophrenia, vascular dementia and other neuro-degenerative conditions are particularly contemplated. Cancerous states, such as breast cancer or hepatoma, also are encompassed.

In another embodiment, the method can be used to identify the complement of proteins within a sample by calculating best filter combinations for determining maximum number of individual peptides after using a particular proteolytic or chemical cleavage technique.

Data produced by the method of the invention can be analysed by sophisticated statistical techniques including uni-variate and multi-variate analysis tools. The following steps can be used to identify target peptide fragments arising from proteins that show an association with a disease or biochemical status:

1. uni-variate differential analysis tools. Changes such as fold changes, Wilcoxon rank sum test and t-test, are useful in determining the significance of the expression values of each target peptide fragment and its corresponding protein of interest.

2. multi-variate differential analysis. The first step is to identify a collection of target peptide fragment signal responses that individually show significant association with any particular condition. The association between the identified proteins and any particular condition need not be as highly significant as is desirable when an individual protein is used in diagnosis.

Once a suitable collection of target peptide fragments has been identified, a sophisticated multi-variate analysis capable of identifying clusters can then be used to estimate the significant multivariate associations with said disease or biochemical status.

Linear Discriminant Analysis (LDA) is one such procedure, which can be used to detect significant association between a cluster of variables and the disease or perturbed biochemical status. In performing LDA, a set of weights is associated with each variable so that the linear combination of weights and the measured values of the variables can identify the disease state by discriminating between subjects having a disease and subjects free from the disease. Enhancements to the LDA allow stepwise inclusion (or removal) of variables to optimize the discriminant power of the model. The result of the LDA is therefore a cluster of target peptide fragments and their corresponding proteins that can be used, without limitation, for diagnosis, prognosis, therapy or drug development. Other enhanced variations of LDA, such as Flexible Discriminant Analysis permit the use of non-linear combinations of variables to discriminate a disease state from a normal state. The results of the discriminant analysis can be verified by post-hoc tests and also by repeating the analysis using alternative techniques such as classification trees.

A further category of proteins of interest can be identified by qualitative measures by comparing the percentage presence of proteins of interest in one group of samples (e.g. samples from diseased subjects) with the percentage presence of a protein of interest in another group of samples (e.g. samples from control subjects). The “percentage presence” of a protein is the percentage of samples in a group of samples in which the protein of interest is detectable by the detection method of choice. For example but without limitation, if a protein of interest is detectable in 95% of samples from diseased subjects, the percentage feature presence of that the protein of interest in that sample group is 95%. If only 5% of samples from non-diseased subjects have detectable levels of the same protein of interest, detection of that protein of interest in the sample of a subject would suggest that it is likely that the subject suffers from the disease. Diagnosis of cancers such as, but not limited to, breast cancer, pancreatic cancer, colorectal cancer or prostate cancer are of particular interest.

The method of the present invention can assist in monitoring a clinical study, e.g. to evaluate drugs for therapy of a disease. For example, candidate molecules can be tested for their ability to restore levels of protein in a diseased subject to levels found in control subjects or, in a treated subject, to preserve levels of protein at normal values. The levels of one or more proteins of interest can be assayed. In another embodiment, the method of the present invention is used to screen candidates for a clinical study to identify individuals having a disease; such individuals can then be either excluded from or included in the study or can be placed in a separate cohort for treatment or analysis.

Many proteins of interest which are associated with various diseases or responses have already been identified such as, but not limited to, those in Table 2.

TABLE 2

| Disease State | Publication No. |
|---|---|
| Breast Cancer | WO 00/55628; WO 01/13117; |
| | WO 01/62914; WO 01/63288; |
| | WO 01/63289; WO 01/63290; |
| | WO 01/71357 |
| Hepatoma | WO 99/41612 |
| | WO 01/13118 |
| Schizophrenia | WO 01/63293 |
| Rheumatoid Arthritis | WO/99/47925 |
| Bipolar Affective Disorder | WO 01/63294 |
| Unipolar Depression | WO 01/63294 |
| Alzheimer's Disease | WO 01/75454; WO 02/46767 |
| Vascular Dementia | WO 01/69261 |
| Kidney disease | WO 02/054081 |
| Vascular cell response | WO 02/054080 |

Results obtained by analyzing proteins in samples of interest can be stored in a database and referenced subsequently. Each new result can be compared with previous results from the same patient allowing the state of the disease to be monitored.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for simplification of explanation. Preferred features of each embodiment of the invention are as for each of the other embodiments mutatis mutandis. All publications, including but not limited to patents and patent applications cited in this specification are herein incorporated by reference as if each individual publication were specifically and individually indicated to be incorporated by reference herein as though fully set forth.

Figure Legends

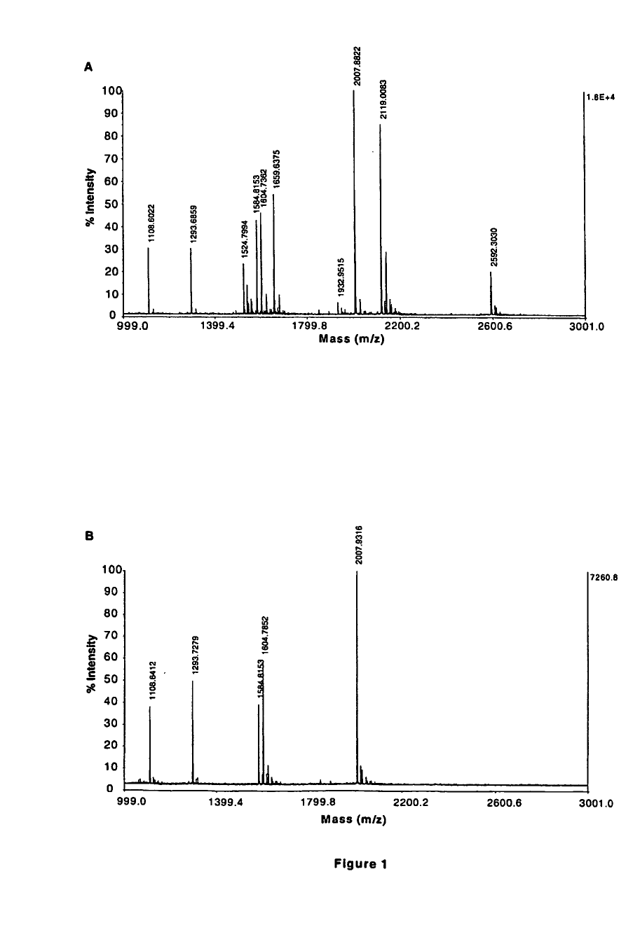

FIG. 1. Quantitative Peptide Depletion Using a Methionine-Reactive Amino Acid Filter

Mass spectra were acquired in the standard reflector mode using a 4700 Proteomics Analyser (Applied Biosystems, Foster City, Calif.). Four hundred laser shots were fired and the resulting mass spectra were averaged to produce each final trace. Panel A shows a spectrum of a peptide sample prepared in the absence of the methionine-reactive amino acid filter (sample A). Panel B is a spectrum of the depleted peptide mixture from an identical peptide sample prepared in the presence of the methionine-reactive amino acid filter (sample B).

### EXAMPLE 1

### Quantitative Peptide Depletion Using a Methionine-Reactive Amino Acid Filter

Peptides were obtained from SIGMA-Genosys. A mixture of 10 synthetic peptides (see Table 3) was used for quantitative peptide depletion using an amino acid filter recognizing methionine (methionine-reactive beads were obtained from The Nest Group, Southborough, Mass., USA). All peptides were biotinylated at their N-terminus.

TABLE 3

List of Peptides for Example 1.

| SEQ | | | Presence of |
|---|---|---|---|
| ID NO. | Peptide Sequence | Mass (m/z) | methionine |
| 1 | RPPQTLSR | 1293.56 | no |
| 2 | NLSPDGQYVPR | 1584.83 | no |
| 3 | SANAEDAQEFSDVER | 2007.13 | no |
| 4 | NFHQYSVEGGK | 1604.82 | no |
| 5 | LERPVR | 1108.38 | no |
| 6 | VFAQNEEIQEMAQNK | 2118.43 | yes |
| 7 | DLPLLIENMK | 1524.92 | yes |
| 8 | ETYGEMADCCAK | 1659.95 | yes |
| 9 | FIMLNLMHETTDK | 1932.37 | yes |
| 10 | DLVTQQLPHLMPSNCGLEEK | 2592.07 | yes |

Preparation of a Methionine Reactive Amino Acid Filter

The met-reactive beads were activated as follows: beads from one “Pi³” isolation pack (approx 10 μl dry settled volume) were washed 5 times, each with 400 μl methanol, followed by 3 washes with 10% (v/v) acetic acid using a spin column. The beads were then resuspended in 400μl 10% (v/v) acetic acid and transferred to a 1.5 ml microcentrifuge tube. The beads were collected by centrifugation and the supernatant (acetic acid) was removed.

Capture of Methionine Containing Peptides on a Met-Reactive Amino Acid Filter

The peptide mixture was prepared as follows: 75 μl of a peptide mixture (Table 3), containing approximately 75 μg peptides in total, was mixed with 25 μl of glacial acetic acid. The peptide mixture was divided equally into two 50 μl aliquots. One aliquot was transferred to the microcentrifuge tubes with the activated met-reactive amino acid filter beads (sample B), whilst another aliquot was incubated without beads (sample A). Samples were incubated at 22° C. for 18 hr. Following incubation, the beads were collected by centrifuging for 1 min at 10,000 rpm in a microcentrifuge. The supernatant was transferred to a fresh tube. This supernatant is called the peptide mixture from sample A or the depleted peptide mixture from sample B.

Mass Spectrometric Analysis

5 μl aliquots were taken from the peptide mixtures A and B (depleted). The volume was then made up to 10 μl in 0.1% (v/v) TFA and the overall amount of TFA adjusted to 0.1%.

Each sample was bound to a ZipTip™, washed in 0.1% TFA and eluted in 1 μl of a solution containing alpha-cyano-4-hydroxycinnamic acid (approximately 2.5 mg/ml in 3:2 methanol:0.1% v/v TFA) and deposited directly onto a target substrate for MALDI-TOF mass spectrometry.

The mass spectrum of peptide mixture sample A (incubated with no beads) is shown in FIG. 1 (Panel A). The ten peaks corresponding to the 10 peptides present in the mixture are indicated by their masses. Peptide mixture sample B (incubated with methionine-reactive beads) was depleted of all Met-containing peptides. The corresponding mass spectrum is shown in FIG. 1 (Panel B). No Met-containing peptides could be detected in the mixture by the mass spectrometry.

1. A method of analysis of a protein mixture, said method comprising:
(a) treating the protein mixture to produce a mixture of peptides;
(b) contacting the mixture of peptides with at least one amino acid filtering agent that binds the side-chain of an amino acid;
(c) depleting the mixture of those peptides that bind to the filtering agent; and
(d) identifying one or more peptides remaining in the depleted mixture.

2. The method according to claim 1, additionally comprising identifying one or more peptides that bind to the amino acid filtering agent.

3. The method according to claim 1, wherein the identification in step (d) comprises mass spectrometry.

4. The method according to claim 3, wherein the identification in step (d) comprises matrix-assisted laser desorption ionisation-time of flight mass spectrometry.

5. The method according to claim 1, wherein step (a) comprises proteolytic digestion of the protein mixture.

6. The method according to claim 5, wherein the proteolytic digestion is performed with trypsin.

7. The method according to claim 1, wherein the amino acid filtering agent covalently binds the side-chain of an amino acid.

8. The method according to claim 1, wherein the amino acid filtering agent binds the side-chain of a naturally occurring amino acid.

9. The method according to claim 1, wherein the amino acid filtering agent is immobilized on a solid support.

10. The method according to claim 1, wherein step (b) comprises contacting the peptide mixture with a plurality of different amino acid filtering agents.

11. The method according to claim 1, wherein step (d) additionally comprises quantifying one or more peptides present in the depleted mixture of peptides and optionally one or more peptides that bind to the amino acid filtering agent.

12. The method according to claim 1, wherein the depleted peptide mixture comprises isotopically labelled peptides.

13. The method according to claim 1, wherein each protein present in the protein mixture is represented by at least one peptide in the depleted peptide mixture.

14. The method according to claim 13, wherein each protein present in the protein mixture is represented by at least three peptides in the depleted peptide mixture.

15. The method according to claim 1, wherein the protein mixture is derived from a biological sample.
